(19) United States
(12) Patent Application Publication
(10) Pub. No.: US 2021/0235967 A1
(43) Pub. Date: Aug. 5, 2021

(54) MEDICAL SYSTEM, NETWORK APPARATUS, MEDICAL APPARATUS, AND INSPECTION INFORMATION PROCESSING METHOD

(71) Applicant: OLYMPUS CORPORATION, Tokyo (JP)

(72) Inventors: Yugo KOIZUMI, Hachioji-shi (JP); Tatsuya SHIOBARA, Hachioji-shi (JP); Kenichiro MATSUSHITA, Hachioji-shi (JP); Yusuke OTSUKA, Hachioji-shi (JP); Yasushi MATSUURA, Hachioji-shi (JP); Haruhiko SAKAYORI, Hachioji-shi (JP); Koichi HIROSE, Hachioji-shi (JP); Yoshinori MATSUMOTO, Hachioji-shi (JP); Takashi OZAKI, Hachioji-shi (JP)

(73) Assignee: OLYMPUS CORPORATION, Tokyo (JP)

(21) Appl. No.: 17/213,730

(22) Filed: Mar. 26, 2021

Related U.S. Application Data
(63) Continuation of application No. PCT/JP2019/012470, filed on Mar. 25, 2019.

(30) Foreign Application Priority Data
Sep. 28, 2018 (JP) 2018-184122

(51) Int. Cl. A61B 1/00; A61B 5/00; G06F 21/62

(52) U.S. Cl. CPC A61B 1/0002; A61B 1/00009; G06F 21/6254; A61B 1/00018; A61B 5/0013; A61B 1/00016

(57) ABSTRACT

A medical system includes a medical apparatus that generates a medical image and a network apparatus communicable with the medical apparatus. The network apparatus generates identification information corresponding to inspection information, stores the inspection information and the identification information in association with each other in a first storage region of a storage apparatus and transmits the identification information to the medical apparatus. The medical apparatus receives the identification information transmitted by the network apparatus and transmits the identification information and the generated medical image in association with each other to the network apparatus. The network apparatus receives the identification information and the medical image associated with each other and transmitted by the medical apparatus and stores the inspection information stored in the first storage region and the medical image in association with each other in a second storage region of the storage apparatus.

### CROSS-REFERENCE TO RELATED APPLICATIONS

This application is based upon and claims the benefit of priority from prior Japanese Patent Application No. 2018-184122, filed Sep. 28, 2018, the entire contents of which are incorporated herein by this reference.

This application is a continuation application of International Application PCT/JP2019/012470 filed on Mar. 25, 2019, the entire contents of which are incorporated herein by reference.

### TECHNICAL FIELD

The present embodiment relates to a medical system provided with a medical apparatus and a network apparatus, the medical apparatus and the network apparatus, and an inspection information processing method executed in the medical system.

### BACKGROUND

Conventional endoscope systems include a function of saving PHI (protected health information) information in a medical apparatus such as a video processor provided for the systems, save images captured during inspections and patient information in association with each other in the medical apparatus and transfer the images and information to a network apparatus such as a server. Here, the PHI information refers to medical information that should be protected defined in HIPAA (Health Insurance Portability and Accountability Act) and the patient information is an example of the PHI information.

As a medical apparatus that prevents leakage of personal information, a medical image diagnostic apparatus disclosed in Japanese Patent Application Laid-Open No. 2011-150699 is known. This apparatus is provided with an input section that inputs personal information of a subject to be inspected, a communication section that communicates with a personal information management server that stores the personal information inputted from the input section, a determination section that determines whether or not the personal information can be transmitted to the personal information management server and an inspection information storage section that stores information of inspection results, in which the communication section transmits the personal information inputted from the input section to the personal information management server based on the determination result of the determination section, receives the personal identification information that identifies the personal information in an unidentifiable manner, returned from the personal information management server in response to the transmission, and the inspection information storage section stores the personal identification information received by the communication section.

### SUMMARY

One aspect of the present embodiment is a medical system including a medical apparatus that generates a medical image and a network apparatus communicable with the medical apparatus, in which the network apparatus includes a first computation device that generates identification information corresponding to inspection information and associates the identification information or the medical image with the inspection information, a storage apparatus including a first storage region and a second storage region, and a first communication interface that communicates with the medical apparatus, the medical apparatus includes a second communication interface that communicates with the network apparatus and a second computation device that generates the medical image, the first computation device stores the inspection information and the identification information in association with each other in the first storage region, the first communication interface transmits the identification information to the medical apparatus, the second communication interface receives the identification information transmitted by the first communication interface and transmits the identification information and the medical image in association with each other to the network apparatus, the first communication interface receives the identification information and the medical image associated with each other and transmitted by the second communication interface, and the first computation device stores the inspection information stored in the first storage region and the medical image in association with each other in the second storage region.

Another aspect of the present embodiment is a medical system including a medical apparatus that generates a medical image and a server communicable with the medical apparatus, in which the server includes a first computation device that generates identification information corresponding to inspection information, a storage apparatus that stores the inspection information and the identification information in association with each other and a first communication interface that communicates with the medical apparatus, the medical apparatus includes a second communication interface that communicates with the server and a second computation device that generates the medical image, the first communication interface transmits the identification information to the medical apparatus, the second communication interface receives the identification information transmitted by the first communication interface, transmits the identification information and the medical image in association with each other to the server, and the first communication interface receives the identification information and the medical image associated with each other and transmitted by the second communication interface.

A further aspect of the present embodiment is a network apparatus communicable with a medical apparatus that generates a medical image, including a computation device that generates identification information corresponding to inspection information and associates the identification information with the inspection information, a storage apparatus that includes a first storage region and a second storage region, and a communication interface that communicates with the medical apparatus, in which the computation device stores the inspection information and the identification information in association with each other in the first storage region, the communication interface transmits the identification information to the medical apparatus, the communication interface receives the identification information and the medical image associated with each other and transmitted by the medical apparatus and the computation device stores the inspection information stored in the first storage region and the medical image in association with each other in the second storage region.

A still further aspect of the present embodiment is a medical apparatus that generates a medical image, including a communication interface that communicates with a network apparatus, a computation device that generates the medical image, in which the communication interface receives identification information corresponding to inspection information transmitted by the network apparatus and transmits the identification information and the medical image in association with each other to the network apparatus.

A still further aspect of the present embodiment is an inspection information processing method executed in a medical system including a medical apparatus that generates a medical image and a network apparatus communicable with the medical apparatus, in which the network apparatus generates identification information corresponding to inspection information, stores the inspection information and the identification information in association with each other in a first storage region of a storage apparatus, transmits the identification information to the medical apparatus, the medical apparatus receives the identification information transmitted by the network apparatus and transmits the identification information and the generated medical image in association with each other to the network apparatus, and the network apparatus receives the identification information and the medical image associated with each other and transmitted by the medical apparatus and stores the inspection information stored in the first storage region and the medical image in association with each other in a second storage region of the storage apparatus.

### BRIEF DESCRIPTION OF DRAWINGS

FIG. 1 is a diagram illustrating a configuration example of a medical system according to a first embodiment;

FIG. 2 is an example of a monitor output image displayed on a monitor;

FIG. 3 is a diagram illustrating an example of a hardware configuration;

FIG. 4 is a flowchart illustrating an example of a processing flow relating to a function according to a second embodiment execute by a server;

FIG. 5 is a flowchart illustrating an example of a processing flow for causing a user to set contents of processing, the processing being executed when a setting in a security setting management section is different among a plurality of video processors, the processing flow is executed by the server; and

FIG. 6 is a diagram illustrating a configuration example of a medical system according to a modification of the first embodiment.

### DESCRIPTION OF EMBODIMENTS

Laws and regulations in recent years demand that PHI information be protected from cyber attacks from outside and means for encrypting data or encrypting communication or the like is required. When vulnerability of OS (operating system) or encryption means is found, a quick response thereto is required.

However, it is not easy to update a medical apparatus and a quick response to laws and regulations is difficult. This is because in the case of a medical apparatus, unlike a consumer product, performance testing or the like needs to be performed to confirm that updating does not affect operation of the medical apparatus or updaters of the medical apparatus are limited to specific trained people.

Therefore, embodiments which will be disclosed hereinafter will provide a medical system, a network apparatus, a medical apparatus, and an inspection information processing method capable of quickly responding to requests for cyber security while maintaining conventional usability.

Hereinafter, embodiments will be described with reference to the accompanying drawings.

### First Embodiment

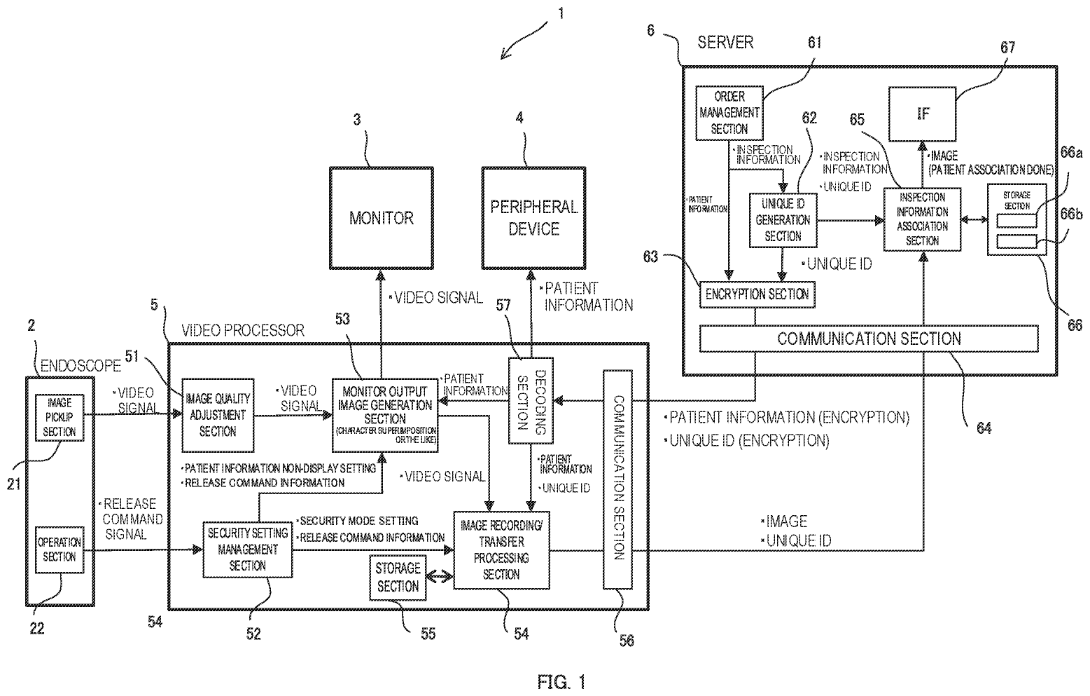

FIG. 1 is a diagram illustrating a configuration example of a medical system according to a first embodiment.

As shown in FIG. 1, a medical system 1 includes a video processor 5 to which an endoscope (also referred to as a “scope”) 2, a monitor 3, and a peripheral device 4 are connected and a server 6. The video processor 5 and the server 6 are connected to a network such as a LAN (local area network). The network is a network constructed of one or both of wired and wireless channels.

The endoscope 2 is provided with an image pickup section 21 and an operation section 22.

The image pickup section 21 is provided with an image pickup device such as a CCD (charge coupled device). The image pickup section 21 picks up images of a subject, which is an imaging object using an image pickup device and outputs a video signal in accordance with the subject.

The operation section 22 includes a release button to give a release instruction, and outputs, for example, a release command signal in response to pressing of the release button. The release command signal is also a recording command signal. In the present embodiment, the release command signal is assumed to be a recording command signal of a still image.

The video processor 5 is an example of the medical apparatus that observes the subject, and is also an example of the endoscope apparatus. The video processor 5 is provided with an image quality adjustment section 51, a security setting management section 52, a monitor output image generation section 53, an image recording/transfer processing section 54, a storage section 55, a communication section 56 (first communication interface), and a decoding section 57.

The decoding section 57 includes a CPU, a memory as a hardware configuration relating to a processing to decode.

The CPU is a computation device that executes a program to perform processing relating to the decoding function.

The memory is a RAM (random access memory) and a ROM (read only memory), and the RAM is used as a work area for the CPU and the ROM stores a program and information necessary to execute the program in a non-volatile manner.

The image quality adjustment section 51 performs image quality adjustment processing on a video signal inputted from the endoscope 2 and outputs the video signal after the image quality adjustment processing.

The security setting management section 52 sets whether or not to display patient information on the monitor 3 for the monitor output image generation section 53. The security setting management section 52 also sets whether or not to record/transfer the patient information for the image recording/transfer processing section 54. These settings are made based on an input operation by the user on an operation panel (not shown) of the video processor 5. That is, the user can freely set whether or not to display the patient information on the monitor 3 or whether or not to record/transfer the patient information. Note that the security setting management section 52 is an example of the storage setting section that can set whether or not to store the patient information in the storage region inside the medical apparatus.

When a release command signal is inputted from the endoscope 2, the security setting management section 52 outputs release command information indicating that a release command is given to the monitor output image generation section 53 and the image recording/transfer processing section 54.

Depending on a setting by the security setting management section 52 and the presence or absence of input of release command information, the monitor output image generation section 53 generates a monitor output image as follows and outputs a video signal indicating the generated monitor output image to the monitor 3 and the image recording/transfer processing section 54.

When a setting that prevents the patient information from being displayed (hereinafter also referred to as “patient information non-display setting”) is made, if release command information is inputted, a monitor output image not including the patient information is generated based on a video signal inputted from the image quality adjustment section 51, and a video signal expressing the monitor output image is outputted to the monitor 3 and the image recording/transfer processing section 54. Note that when a monitor output image without including the patient information is generated, character information other than the patient information and an endoscopic image expressed by the video signal are subjected to superimposition processing or the like. Accordingly, when the patient information non-display setting is made, if the release button is pressed, a video signal not including the patient information is outputted.

On the other hand, when the patient information non-display setting is made and the release command information is not inputted or when a setting that the patient information is displayed (hereinafter also referred to as “patient information display setting”) is made, a monitor output image including the patient information is generated based on the video signal inputted from the image quality adjustment section 51 and the patient information inputted from the decoding section 57, and a video signal expressing the monitor output image is outputted to the monitor 3 and the image recording/transfer processing section 54. Note that when the monitor output image including the patient information is generated, character information including the patient information and the endoscopic image expressed by the video signal are subjected to superimposition processing. Accordingly, a video signal including the patient information is outputted when the patient information non-display setting is made and the release button is not pressed or when the patient information display setting is made.

Note that the monitor output image is an example of a medical image regarding the subject and the monitor output image generation section 53 is an example of the display control section that causes the display apparatus to display a display image including the medical image and the patient information.

Upon receiving the release command information, the image recording/transfer processing section 54 records a unique ID (identifier) or patient information and the monitor output image in association with each other in the storage section 55 in accordance with the setting by the security setting management section 52 as follows and transfers the unique ID (identifier) or patient information and the monitor output image associated with each other to the server 6.

When a setting that prevents the patient information from being recorded/transferred (hereinafter also referred to as “security mode setting”) is made, if release command information is inputted, the unique ID inputted from the decoding section 57 is associated with the monitor output image expressed by the video signal inputted from the monitor output image generation section 53 and recorded in the storage section 55 and transferred to the server 6. Accordingly, when a security mode is set, if the release button is pressed, the unique ID is associated with the monitor output image, recorded in the storage section 55 and transferred to the server 6.

On the other hand, when a setting that causes the patient information to be recorded/transferred is made, if the release command information is inputted, the patient information inputted from the decoding section 57 is associated with the monitor output image expressed by the video signal inputted from the monitor output image generation section 53, recorded in the storage section 55 and transferred to the server 6. Accordingly, when the setting that causes the patient information to be recorded/transferred is made, if the release button is pressed, the patient information is associated with the monitor output image, recorded in the storage section 55 and transferred to the server 6.

The storage section 55 is a non-volatile storage apparatus, for example, a hard disk apparatus. The storage section 55 records the monitor output image and unique ID or patient information associated with each other.

The communication section 56 is a communication interface that communicates with an external apparatus such as the server 6.

The first communication interface 56 of the video processor 5 and the second communication interface 64 of the server 6 are connected via the network (Not shown in the figure.).

Network 4 is a communication channel using, for example, the Internet or a wireless LAN, and can include a dedicated line (dedicated cable) for direct connection, a LAN using Ethernet (registered trademark), etc., as well as a communication network such as a telephone communication network, cable network, or wireless LAN.

The communication method can be wired or wireless.

The communication section 56 transmits, for example, the monitor output image, the unique ID or the patient information associated with each other and transferred by the image recording/transfer processing section 54 to the server 6. The communication section 56 receives, for example, the unique ID and patient ID transmitted and encrypted by the server 6.

The decoding section 57 decodes the encrypted unique ID and patient ID received by the communication section 56, outputs the unique ID and patient information to the image recording/transfer processing section 54 and outputs the patient information to the monitor output image generation section 53 and the peripheral device 4.

The monitor 3 is an example of the display apparatus and is, for example, an LCD (liquid crystal display). The monitor 3 displays a monitor output image expressed by the video signal inputted from the monitor output image generation section 53.

The peripheral device 4 is, for example, a printing apparatus that prints the patient information outputted by the decoding section 57.

The server 6 is an example of the network apparatus communicable with the medical apparatus. The server 6 is provided with an order management section 61, a unique ID generation section 62, an encryption section 63, a communication section 64 (second communication interface), an inspection information association section 65, a storage section 66, and an IF (interface) 67. The order management section 61 performs centralized data management on inspection order information by storing the inspection order information. The inspection order information includes inspection information. The inspection information includes patient information and information relating to an inspection date and an inspection type or the like. The patient information includes information relating to the patient ID and the name of a patient or the like. According to the user's input operation on, for example, the server 6, the order management section 61 outputs inspection information included in the inspection order information relating to endoscopy to be executed next to the unique ID generation section 62 and outputs patient information included in the inspection information to the encryption section 63.

The unique ID generation section 62 generates a unique ID corresponding to the inspection information outputted by the order management section 61. Note that the unique ID is also identification information of the inspection information. The unique ID generation section 62 outputs the generated unique ID and the inspection information to the inspection information association section 65 and outputs the generated unique ID to the encryption section 63.

The encryption section 63 encrypts the patient information outputted by the order management section 61 and the unique ID outputted by the unique ID generation section 62.

The communication section 64 is a communication interface that communicates with an external apparatus such as the video processor 5. The communication section 64 transmits the patient information and the unique ID encrypted by the encryption section 63 to the video processor 5. The communication section 64 receives the monitor output image and the unique ID or the patient information associated with each other and transmitted by the video processor 5.

The inspection information association section 65 stores the unique ID and the inspection information outputted by the unique ID generation section 62 in association with each other in a first storage region 66a of the storage section 66. The inspection information association section 65 reads the inspection information corresponding to the unique ID or the patient information associated with the monitor output image received by the communication section 64 from the first storage region 66a of the storage section 66, and stores the read inspection information and the monitor output image in association with each other in a second storage region 66b. The inspection information association section 65 can also output the associated inspection information and the monitor output image to the IF 67 and store the associated inspection information and the monitor output image in an external storage apparatus such as an EMR (electronic medical record) connected to the IF 67.

The storage section 66 is a non-volatile storage apparatus and is, for example, a hard disk apparatus. As described above, the storage section 66 includes the first storage region 66a that stores the unique ID in association with the inspection information and the second storage region 66b that stores the inspection information in association with the monitor output image.

The IF 67 is a connection interface to which an external storage apparatus such as an EMR is connected.

Next, processes executed by the medical system 1 will be described.

In the medical system 1, according to the user's input operation, for example, on the video processor 5, the security setting management section 52 sets whether or not to display the patient information on the monitor 3 for the monitor output image generation section 53 and also sets whether or not to record/transfer the patient information for the image recording/transfer processing section 54.

In the medical system 1, according to the user's input operation, for example, on the server 6, the order management section 61 outputs inspection information included in the inspection order information relating to endoscopy to be executed next to the unique ID generation section 62 and outputs the patient information included in the inspection information to the encryption section 63 and executes the next processing.

Upon acquiring the inspection information outputted by the order management section 61, the unique ID generation section 62 generates a unique ID corresponding to the inspection information, outputs the unique ID and the inspection information to the inspection information association section 65 and also outputs the unique ID to the encryption section 63.

Upon acquiring the unique ID and the inspection information outputted by the unique ID generation section 62, the inspection information association section 65 stores the unique ID and the inspection information in association with each other in the first storage region 66a of the storage section 66.

Upon acquiring the patient information outputted by the order management section 61 and the unique ID outputted by the unique ID generation section 62, the encryption section 63 encrypts the patient information and the unique ID. The communication section 64 transmits the encrypted patient information and unique ID to the video processor 5.

In the video processor 5, when the communication section 56 receives the encrypted patient information and unique ID transmitted by the server 6, the decoding section 57 decodes the encrypted patient information and unique ID, outputs the patient information and the unique ID to the image recording/transfer processing section 54 and outputs the patient information to the monitor output image generation section 53 and the peripheral device 4. For example, a printing apparatus, which is the peripheral device 4 prints the patient information outputted by the decoding section 57.

After that, when endoscopy related to the inspection information outputted by the order management section 61 starts, the image quality adjustment section 51 in the video processor 5 performs image quality adjustment processing on the video signal inputted from the endoscope 2 and starts processing of outputting the video signal to the monitor output image generation section 53. When a release command signal is inputted from the endoscope 2, the security setting management section 52 outputs the release command information to the monitor output image generation section 53 and the image recording/transfer processing section 54.

Depending on the setting by the security setting management section 52 or the presence or absence of input of release command information, the monitor output image generation section 53 generates a monitor output image as described above and outputs a video signal expressing the generated monitor output image to the monitor 3 and the image recording/transfer processing section 54.

The monitor 3 displays the monitor output image expressed by the video signal inputted from the monitor output image generation section 53.

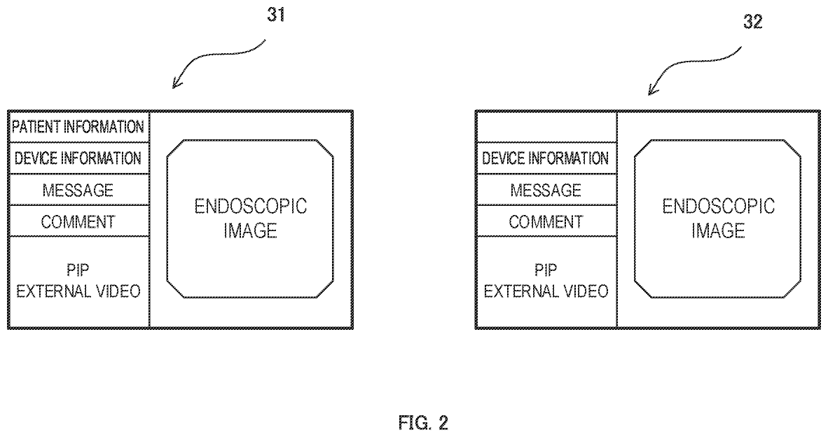

FIG. 2 is a diagram illustrating an example of a monitor output image displayed on the monitor 3.

In FIG. 2, a monitor output image 31 shown on the left is a monitor output image displayed on the monitor 3 when the setting in the monitor output image generation section 53 is a patient information display setting or when the setting in the monitor output image generation section 53 is a patient information non-display setting and the release button is not pressed, and is a monitor output image including the patient information.

On the other hand, a monitor output image 32 shown on the right is a monitor output image displayed on the monitor 3 when the setting in the monitor output image generation section 53 is a patient information non-display setting and the release button is pressed, and is a monitor output image not including the patient information.

Upon receiving the release command information, the image recording/transfer processing section 54 performs processing of recording the unique ID or the patient information and the monitor output image in association with each other in the storage section 55 and transferring the unique ID or the patient information and the monitor output image associated with each other to the server 6 according to the setting by the security setting management section 52. The transfer to the server 6 is performed by transmission to the server 6 by the communication section 56.

In the server 6, when the communication section 64 receives the unique ID or the patient information and the monitor output image associated with each other and transmitted by the video processor 5, the inspection information association section 65 reads the inspection information corresponding to the unique ID or the patient information associated with the monitor output image from the first storage region 66a of the storage section 66 and stores the read inspection information and the monitor output image in association with each other in the second storage region 66b. The inspection information association section 65 may output the associated inspection information and monitor output image to the IF 67 and store the associated inspection information and monitor output image in an external storage apparatus such as an EMR connected to the IF 67.

When the endoscopy ends and the power switch of the video processor 5 is turned off, the video processor 5 erases all the patient information stored in a storage region in the own apparatus such as the storage section 55.

As described so far, according to the present embodiment, it is possible to prevent patient information from being recorded in the video processor by making a patient information non-display setting on the monitor output image generation section 53 and making a security mode setting on the image recording/transfer processing section 54. The server 6 can associate a monitor output image generated by the video processor 5 with the inspection information including the patient information using a unique ID, which is not PHI information.

In this case, the video processor 5 does not save the patient information, which is PHI information, and so the video processor 5 does not possess data to be protected from a cyber attack, which reduces requirements for cyber security. On the other hand, since the server 6 records the patient information or the like, and thereby responds to the requirements relating to cyber security, but updating of the server 6 is easier compared to the video processor 5, which is a medical instrument, and so a quick response is possible.

According to the present embodiment, it is possible to display patient information or record/transfer the patient information as before by making a patient information display setting for the monitor output image generation section 53 and making a setting of recording/transferring the patient information for the image recording/transfer processing section 54. Therefore, it is possible to maintain conventional usability. It is also possible to output the patient information to the peripheral device 4 as before.

Note that various modifications can be made in the present embodiment.

For example, when a patient information non-display setting is made in the monitor output image generation section 53, a configuration may also be adopted in which a monitor output image not including patient information is generated uniformly regardless of the presence or absence of input of the release command information and a video signal expressing the monitor output image is outputted to the monitor 3 and the image recording/transfer processing section 54. This makes it possible to output a video signal not including patient information uniformly when the patient information non-display setting is made.

When, for example, a patient information non-display setting is made in the monitor output image generation section 53, a monitor output image not including the patient information is generated during a period after an endoscopy start is instructed until an endoscopy end is instructed and a video signal expressing the monitor output image is outputted to the monitor 3 and the image recording/transfer processing section 54. This makes it possible to output a video signal not including patient information only during an inspection when the patient information non-display setting is made.

For example, when the patient information non-display setting is made in the monitor output image generation section 53, a configuration may be adopted in which a monitor output image not including patient information is generated only during a period after a moving recording start is instructed until a moving recording end is instructed and a video signal expressing the monitor output image is outputted to the monitor 3 and the image recording/transfer processing section 54. This makes it possible to output a video signal not including patient information only during moving image recording. In this case, a configuration may be adopted in which the monitor output image to be recorded/transferred by the image recording/transfer processing section 54 is designated as a moving image based on the video signal inputted from the monitor output image generation section 53 during a period after the moving image recording start is instructed until a moving image recording end is instructed.

For example, the decoding section 57 may be omitted from the video processor 5 and a decoding apparatus communicable with the video processor 5 and the server 6 may be provided between the two instead. In this case, the decoding apparatus receives the encrypted patient information and unique ID transmitted by the server 6, decodes the encrypted patient information and unique ID and transmits the patient information and unique ID to the video processor 5. The communication section 56 in the video processor 5 receives the patient information and unique ID transmitted by the decoding apparatus, outputs the patient information and unique ID to the image recording/transfer processing section 54 and outputs the patient information to the monitor output image generation section 53 and the peripheral device 4. When vulnerability of the encryption is found, this makes it possible to solve the vulnerability only by updating the decoding apparatus and the server 6.

For example, the medical system 1 may be configured such that inspection order information managed by the server 6 can be searched according to the user's input operation on the video processor 5. For example, the medical system 1 may also be configured such that the inspection order information relating to the next endoscopy is instructed to the server 6 according to the user's input operation on the video processor 5. The medical system 1 may also be configured, for example, such that the video processor 5 acquires information relating to the inputted patient ID from the server 6 when the user inputs the patient ID to the video processor 5.

In consideration of a case where the power of the video processor 5 is turned off due to, for example, replacement of the endoscope 2 during endoscopy, unless the unique ID is overwritten, that is, unless the next unique ID is acquired from the server 6, the video processor 5 maintains the unique ID and performs processing by the image recording/transfer processing section 54 using the unique ID possessed.

For example, the medical system 1 may also be configured so as not to transmit the patient information from the server 6 to the video processor 5. In this case, the medical system 1 may be configured such that the encryption section 63 is omitted from the server 6, the decoding section 57 is omitted from the video processor 5 and the unique ID is transmitted without being encrypted from the server 6 to the video processor 5. In this case, the communication section 64 transmits the unique ID and the communication section 56 receives the unique ID. A configuration may also be adopted in which the security setting management section 52 makes no setting for the monitor output image generation section 53 and the image recording/transfer processing section 54. In this case, regarding the generation of a monitor output image, the monitor output image generation section 53 may be configured to generate only a monitor output image not including the patient information, and regarding the association with the monitor output image, the image recording/transfer processing section 54 may be configured only to associate the monitor output image with the unique ID.

For example, the image quality adjustment section 51, the security setting management section 52, the monitor output image generation section 53, the image recording/transfer processing section 54 and the decoding section 57 in the video processor 5 may also be implemented using a circuit such as an FPGA (field-programmable gate array) or ASIC (application specific integrated circuit).

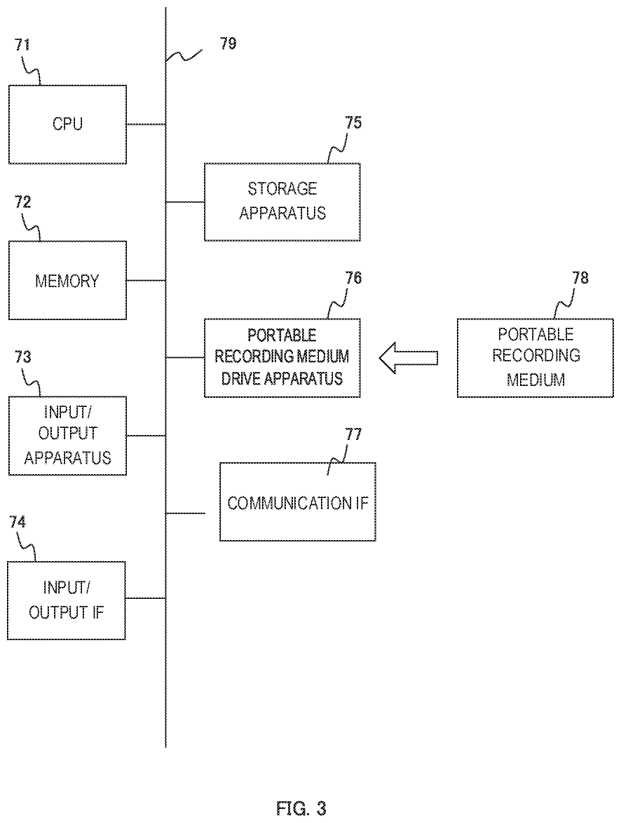

For example, the video processor 5 may also be implemented by a hardware configuration illustrated in FIG. 3.

FIG. 3 is a diagram illustrating an example of the hardware configuration.

The hardware configuration shown in FIG. 3 is constructed of a CPU 71, a memory 72, an input/output apparatus 73, an input/output IF 74, a storage apparatus 75, a portable recording medium drive apparatus 76 for storing a portable recording medium 78, and a communication IF 77, all of which are connected to each other via a bus 79.

The CPU 71 is a computation device that executes a program for processing carried out by the video processor 5. The memory 72 is a RAM and a ROM, the RAM is used as a work area or the like of the CPU 71 and the ROM stores, in a non-volatile manner, a program and information necessary to execute the program.

The input/output apparatus 73 is constructed of input apparatuses such as an operation panel, a touch panel, or a keyboard, and output apparatuses such as a display panel.

The input/output IF 74 is an interface for transmitting/receiving signals to/from the endoscope 2, the monitor 3, the peripheral device 4, or the like.

The storage apparatus 75 is a storage that stores a program, information necessary to execute the program, and information acquired by executing the program in a non-volatile manner. The storage apparatus 75 is, for example, a hard disk apparatus. The portable recording medium drive apparatus 76 drives the portable recording medium 78 to access recording contents thereof. Examples of the portable recording medium 78 include a memory device, a flexible disk, an optical disk, or a magneto-optical disk. Examples of the portable recording medium 78 include a CD-ROM (compact disk read only memory), a DVD (digital versatile disk), a USB (universal serial bus) memory. The portable recording medium 78 is also a storage that stores a program, information necessary to execute the program and information acquired by executing the program in a non-volatile manner, like the storage apparatus 75.

The communication IF 77 is an interface connected to a network and for communication with an external apparatus such as the server 6.

For example, the server 6 may also be implemented in the hardware configuration shown in FIG. 3. In this case, in the hardware configuration shown in FIG. 3, the CPU 71 is a computation device that executes a program for executing processing carried out by the server 6. The input/output apparatus 73 is constructed of input apparatuses such as a keyboard, a mouse, or a touch panel, and output apparatuses such as a display apparatus. The input/output IF 74 is an interface for transmitting/receiving signals to/from an external storage apparatus such as an EMR. The communication IF 77 is an interface connected to a network for communication with an external apparatus such as the video processor 5.

### Second Embodiment

Next, a second embodiment will be described.

In the second embodiment, the same components as those in the first embodiment will be described with the same reference numerals.

In the medical system 1 according to the second embodiment, a plurality of video processors 5 are connected to a network and the server 6 is communicably connected to each video processor 5 via a network. As shown, for example, in FIG. 1, each video processor 5 is connected to the endoscope 2, the monitor 3, and the peripheral device 4.

As described in the first embodiment, each video processor 5 allows the user to freely set whether or not to cause the monitor output image generation section 53 to display patient information on the monitor 3 and whether or not to cause the image recording/transfer processing section 54 to record/transfer patient information. That is, the user can set whether or not to cause the video processor 5 to record patient information.

On the other hand, whether or not to cause the video processor 5 to record the patient information depends on the user's setting, and so, there needs to be a mechanism that grasps setting situations in the plurality of video processors 5 and takes appropriate action as required in terms of security.

Thus, in the medical system 1 according to the second embodiment, the server 6 further includes the following functions: A function to acquire from each video processor 5, a setting in the security setting management section 52 on the monitor output image generation section 53 and the image recording/transfer processing section 54 (hereinafter referred to as “setting in the security setting management section 52”); a function to determine whether or not the setting by the security setting management section 52 is different among the plurality of video processor 5; and a function, when the setting in the security setting management section 52 is different among the plurality of video processor 5, to perform any one or more of announcement, notification, and communication interruption.

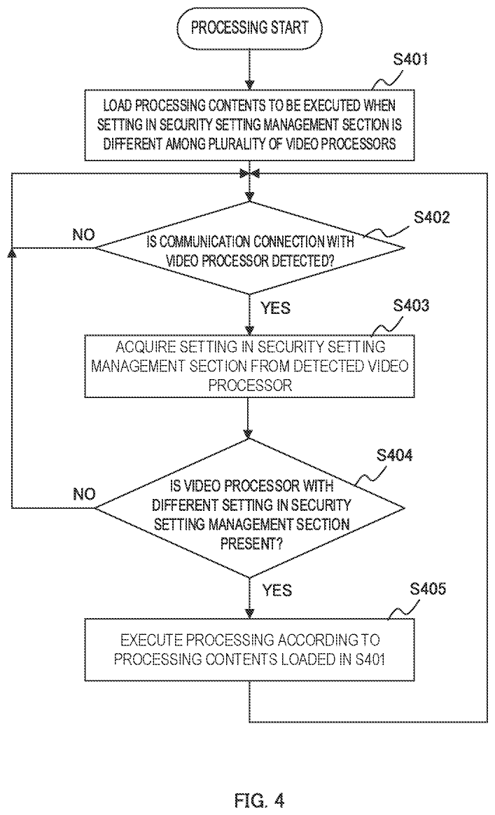

FIG. 4 is a flowchart illustrating an example of a processing flow relating to the above-described functions executed by the server 6.

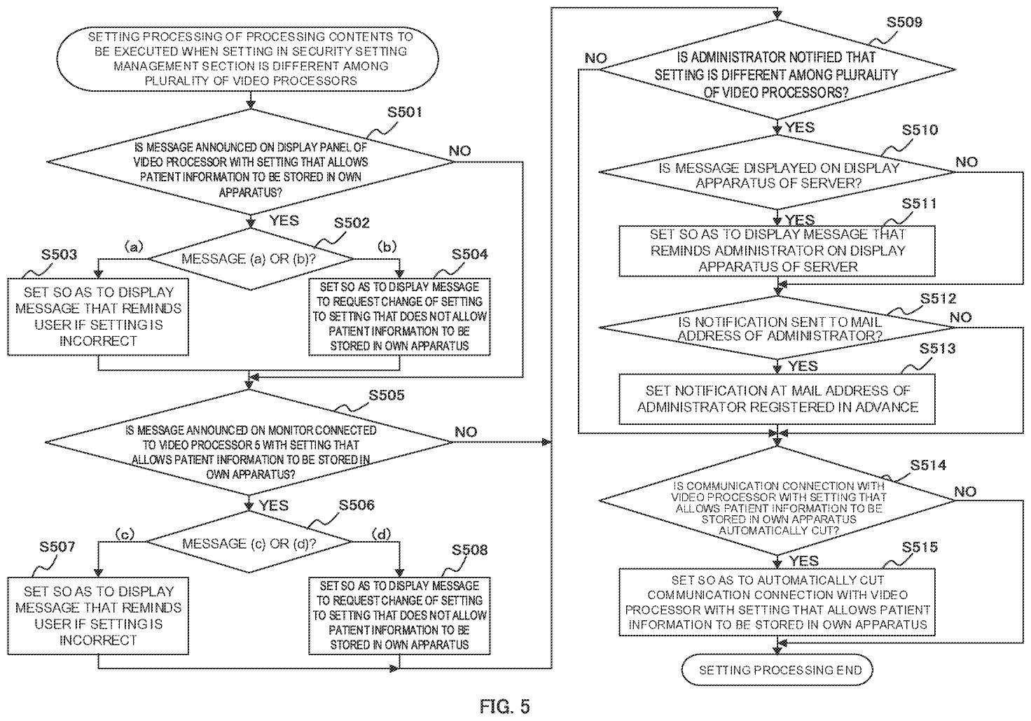

As shown in FIG. 4, first in S401, the server 6 loads processing contents supposed to be executed when a determination result in S404, which will be described later, is YES in this process. Note that the case where the determination result in S404 is YES refers to a case where the setting in the security setting management section 52 is different among the plurality of video processors 5 as will be described later. The processing contents to be loaded depend on the execution of a process shown in FIG. 5, which will be described later, and are set in the server 6 under the choice of the user.

Next, in S402, the server 6 determines whether or not a communication connection with the video processor 5 is detected.

When the determination result in S402 is NO, the present determination is repeated.

On the other hand, when the determination result in S402 is YES, in S403, the server 6 acquires the setting in the security setting management section 52 from the video processor 5 that has detected the communication connection. For example, when communication connections with the plurality of video processors 5 are detected in S402, the settings in the security setting management section 52 are acquired from the respective video processors 5.

In S404, the server 6 determines whether or not there are video processors 5, the setting in the security setting management section 52 of which is different among the plurality of video processors 5 from which the communication connection is detected in S402. Note that in S402 when only one video processor 5 has detected the communication connection, the determination result in S404 is processed as NO.

When the determination result in S404 is NO, the flow returns to S402.

On the other hand, when the determination result in S404 is YES, in S405, the server 6 executes processing according to the processing contents loaded in S401 and then returns to S402.

FIG. 5 is a flowchart illustrating an example of a processing flow for causing the user to set contents of processing, the processing being executed when the setting in the security setting management section 52 is different among the plurality of video processors 5, the processing flow is executed by the server 6.

As shown in FIG. 5, first in S501, the server 6 causes the user to choose whether or not to announce a message on the display panel of the video processor 5, a setting in the security setting management section 52 according to which the patient information is stored in the own apparatus. Note that the setting according to which the patient information is stored in the own apparatus is a patient information display setting for the monitor output image generation section 53 and/or a setting that allows the patient information to be recorded/transferred for the image recording/transfer processing section 54.

When the user's choice in S501 is NO, the flow proceeds to S505.

On the other hand, when the user's choice in S501 is YES, in S502, the server 6 causes the user to choose message (a) or (b) to be announced on the display panel of the video processor 5. Message (a) is a message that reminds the user if the setting in the security setting management section 52 is incorrect. Message (b) is a message that requests the user to change the setting in the security setting management section 52 to a setting that does not allow the patient information to be stored in the own apparatus. Note that the “setting that does not allow the patient information to be stored in the own apparatus” means a patient information non-display setting for the monitor output image generation section 53 and a security mode setting for the image recording/transfer processing section 54.

When the user's choice in S502 is (a), the server 6 makes a setting to execute processing of displaying message (a) on the display panel of the video processor 5 in S503 and proceeds to S505.

On the other hand, when the user's choice in S502 is (b), the server 6 makes a setting to execute processing of displaying message (b) on the display panel of the video processor 5 in S504 and proceeds to S505.

In S505, the server 6 causes the user to choose whether or not to announce a message on the monitor 3 connected to the video processor 5 in which the setting in the security setting management section 52 is a setting according to which patient information is stored in the own apparatus.

When the user's choice in S505 is NO, the flow proceeds to S509.

On the other hand, when the user's choice in S505 is YES, the server 6 causes the user to choose message (c) or (d) to be announced by the monitor 3 connected to the video processor 5 in S506. Message (c) is the same as above-described message (a). Message (d) is the same as above-described message (b).

When the user's choice in S506 is (c), the server 6 makes a setting to execute processing of displaying message (c) on the monitor 3 connected to the video processor 5 in S507 and the flow proceeds to S509.

On the other hand, when the user's choice in S506 is (d), the server 6 makes a setting to execute processing of displaying message (d) on the monitor 3 connected to the video processor 5 in S508 and the flow proceeds to S509.

In S509, the server 6 causes the user to choose whether or not to notify an administrator that the setting in the security setting management section 52 is different among the plurality of video processors 5. Here, the administrator is, for example, an administrator of the medical system 1 or an administrator of the server 6.

When the user's choice in S509 is NO, the flow proceeds to S514.

On the other hand, when the user's choice in S509 is YES, the server 6 causes the user to choose whether or not to display a message on the display apparatus provided in the server 6 as a notification to the administrator in S510. Note that a display apparatus of a terminal apparatus connected to the server 6 via a network may be used instead of the display apparatus provided for the server 6.

When the user's choice in S510 is NO, the flow proceeds to S512.

On the other hand, when the user's choice in S510 is YES, the server 6 makes a setting so as to execute processing of displaying a message that reminds the administrator on the display apparatus of the server 6 as a notification to the administrator and the flow proceeds to S512.

In S512, the server 6 causes the user to choose whether or not to notify to an email address of the administrator as a notification to the administrator.

When the user's choice in S512 is NO, the flow proceeds to S514.

On the other hand, when the user's choice in S512 is YES, in S513, the server 6 makes a setting to execute processing of notification to the email address of the administrator registered in advance as a notification to the administrator and the flow proceeds to S514.

In S514, the server 6 causes the user to choose whether or not to automatically cut a communication connection with the video processor 5 with the setting in the security setting management section 52 that the patient information is stored in the own apparatus.

When the user's choice in S514 is NO, the processing shown in FIG. 5 ends.

On the other hand, when the user's choice in S514 is YES, in S515, the server 6 makes a setting to execute the processing of automatically cutting a communication connection with the video processor 5 with the setting in the security setting management section 52 that the patient information is stored in the own apparatus and the processing shown in FIG. 5 ends.

As described so far, according to the second embodiment, when the setting in the security setting management section 52 of the medical system 1 is different among the plurality of video processors 5, it is possible to perform any one or more of the above-described announcement, notification, and communication interruption. This promotes that the settings in the security setting management sections 52 among the plurality of video processors 5 are unified into such a setting that the patient information is not stored in the own apparatus, and it is thereby possible to further reduce the risk of leakage of the patient information in the event that the video processor 5 receives a cyber attack.

The first and second embodiments have been described so far.

A video processor, which is an example of an endoscope apparatus, has been described as an example of the medical apparatus in the respective embodiments, but another medical imaging apparatus such as an ultrasonic observation device may also be adopted as another example of the medical apparatus.

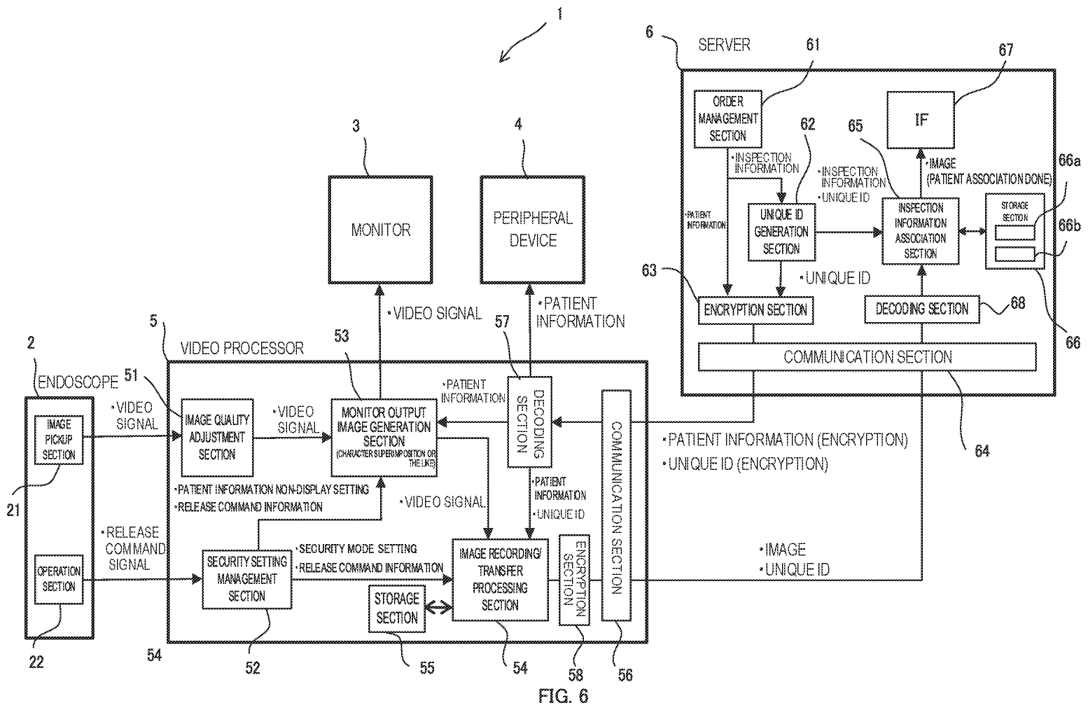

In the first embodiment, the configuration of the endoscope system 1 may be modified as shown in FIG. 6 so that data transferred from the video processor 5 to the server 6 is encrypted.

FIG. 6 is a diagram illustrating a configuration example of an endoscope system 1 according to a modification of the first embodiment.

The endoscope system 1 shown in FIG. 6 is different from the endoscope system 1 shown in FIG. 1 in that the video processor 5 is further provided with an encryption section 58 and the server 6 is further provided with a decoding section 68.

The encryption section 58 is provided between the image recording/transfer processing section 54 and the communication section 56 and configured to encrypt data transferred by processing of the image recording/transfer processing section 54. The encrypted data is transmitted to the server 6 by the communication section 56.

The decoding section 68 is provided between the communication section 64 and the inspection information association section 65, and when the communication section 64 receives the encrypted data, the decoding section 68 decodes the encrypted data and outputs the decoded data to the inspection information association section 65.

This causes data transferred from the video processor 5 to the server 6, that is, the associated unique ID or patient information and monitor output image to be encrypted and the data can thereby be protected.

In the first embodiment, the storage section 66 of the server 6 may further store the monitor output image and the unique ID associated with the monitor output image. In this case, for example, the storage section 66 may further include a third storage region and store the monitor output image and the unique ID associated with the monitor output image in the third storage region.

The present embodiment is not limited to the above-described embodiments as is, but the components thereof may be modified and embodied without departing from the gist of the present invention in an implementation stage. Furthermore, various embodiments may be formed by combining the plurality of components disclosed in the above-described embodiments as appropriate. For example, some of all the components disclosed in the embodiments may be deleted. Furthermore, components across different embodiments may be combined as appropriate.

1. A medical system comprising a medical apparatus including a video processor configured to generate a medical image and a network apparatus communicable with the medical apparatus, wherein
the network apparatus comprises:
a first computation device including a processor configured to generate identification information corresponding to inspection information and associate the identification information or the medical image with the corresponding inspection information;
a storage apparatus comprising a first storage region and a second storage region; and
a first communication interface configured to communicate with the medical apparatus,
the medical apparatus comprises:
a second communication interface configured to communicate with the network apparatus; and
a second computation device including a processor configured to generate the medical image,
the first computation device including a memory configured to store the inspection information and the identification information in association with each other in the first storage region,
the first communication interface configured to transmit the identification information to the medical apparatus,
the second communication interface configured to receive the identification information transmitted by the first communication interface and transmit the identification information and the medical image in association with each other to the network apparatus,
the first communication interface configured to receive the identification information and the medical image associated with each other and transmitted by the second communication interface, and
the first computation device configured to store the inspection information stored in the first storage region and the medical image in association with each other in the second storage region.

2. The medical system according to claim 1, wherein
the first computation device configured to further encrypt patient information included in the inspection information and the identification information,
the first communication interface configured to transmit the encrypted patient information and identification information to the medical apparatus,
the medical apparatus is an endoscope apparatus to which an endoscope is connected, and
the second computation device further displays a display image including the medical image and the patient information on a display apparatus.

3. The medical system according to claim 2, wherein
the second communication interface receives the encrypted patient information and identification information transmitted by the first communication interface, and
the second computation device decodes the encrypted patient information and identification information.

4. The medical system according to claim 2, further comprising a decoding apparatus that includes a processor configured to:
receive the encrypted patient information and identification information transmitted by the first communication interface; and
decode the encrypted patient information and identification information and transmits the patient information and the identification information to the medical apparatus, wherein:
the second communication interface receives the patient information and the identification information transmitted by the decoding apparatus.

5. The medical system according to claim 2, wherein
the second computation device can further set whether or not to store the patient information in a storage region in an own apparatus.

6. The medical system according to claim 5, wherein
the medical apparatus erases the patient information from the storage region in the own apparatus after the inspection by the endoscope ends.

7. The medical system according to claim 6, wherein
the network apparatus is configured to:
connect and communicate with a plurality of the medical apparatuses;
acquire setting contents in the second computation device of each medical apparatus; and
notify a user of a case where the setting contents in the plurality of the medical apparatuses are a mixture of setting contents of the patient information that is stored in the own apparatus and setting contents of the patient information that is not stored in the own apparatus.

8. The medical system according to claim 1, wherein
the first computation device further stores the associated inspection information and medical image in an external storage apparatus connected to the network apparatus.

9. A medical system comprising a medical apparatus including a video processor configured to generate a medical image and a server configured to communicate with the medical apparatus, wherein
the server comprises:
a first computation device that includes a processor configured to generate identification information corresponding to inspection information; and
a storage apparatus including a memory configured to store the inspection information and the corresponding identification information; and
a first communication interface configured to communicate with the medical apparatus,
the medical apparatus comprises:
a second communication interface configured to communicate with the server; and
a second computation device configured to generate the medical image,
the first communication interface transmits the identification information to the medical apparatus,
the second communication interface receives the identification information transmitted by the first communication interface, transmits the identification information and the medical image associated with the identification information to the server, and
the first communication interface receives the identification information and the medical image associated with the identification information and transmitted by the second communication interface.

10. The medical system according to claim 9, wherein
the storage apparatus stores the inspection information and the medical image in association with each other.

11. The medical system according to claim 10, wherein
the storage apparatus further stores the medical image and the identification information.

12. A network apparatus communicable with a medical apparatus including a video processor configured to generate a medical image, comprising:
a computation device that includes a processor configured to generate identification information corresponding to inspection information and associates the identification information with the inspection information;
a storage apparatus including a memory that comprises a first storage region and a second storage region; and
a communication interface that communicates with the medical apparatus, wherein
the computation device stores the inspection information and the identification information in association with each other in the first storage region,
the communication interface transmits the identification information to the medical apparatus,
the communication interface receives the identification information and the medical image associated with each other and transmitted by the medical apparatus, and
the computation device including a memory configured to store the inspection information stored in the first storage region and the medical image in association with each other in the second storage region.

13. A medical apparatus that generates a medical image, comprising:
a communication interface configured to communicate with a network apparatus; and
a computation device including a processor configured to generate the medical image, wherein
the communication interface configured to receive identification information corresponding to inspection information transmitted by the network apparatus and transmits the identification information and the medical image in association with each other to the network apparatus.

14. An inspection information processing method executed in a medical system comprising a medical apparatus including a video processor configured to generate a medical image and a network apparatus configured to communicate with the medical apparatus, wherein
the network apparatus generates identification information corresponding to inspection information, stores the inspection information and the corresponding identification information in a first storage region of a storage apparatus, and transmits the identification information to the medical apparatus,
the medical apparatus receives the identification information transmitted by the network apparatus and transmits the identification information and the generated medical image associated with the identification information to the network apparatus, and
the network apparatus receives the identification information and the medical image associated with the identification information and transmitted by the medical apparatus and stores the inspection information stored in the first storage region and the medical image associated with the inspection information in a second storage region of the storage apparatus.
